(19) United States
(12) Patent Application Publication
(10) Pub. No.: US 2005/0244461 A1
(43) Pub. Date: Nov. 3, 2005

(54) Controlled release drug delivery systems and methods for treatment of an eye

(75) Inventors: Thierry Nivaggioli, Los Altos Hills, CA (US); Wendy Blanda, Laguna Niguel, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA (US)

(21) Appl. No.: 10/836,904

(22) Filed: Apr. 30, 2004

(51) Int. Cl. A61K 9/00

(52) U.S. Cl. 424/427

(57) ABSTRACT

Systems and method are provided for treatment of an eye. The systems generally include controlled release implantable elements including a therapeutic component and a substantially inactive matrix component. The systems include such elements having controlled porosities and/or controlled surface roughness. The elements are typically bioerodible and structured to be implantable into a desired location of an eye to provide delivery of the therapeutic component to the eye. The elements exhibit relatively more controllable, more predictable, drug release rate profile in comparison to substantially identical elements without such controlled porosities and/or surface roughness.

The present invention generally relates to drug delivery systems for controlled, sustained and/or delayed drug release in eyes, and more specifically relates to controlled release drug delivery implants and methods of using such implants, for treatment of eyes, for example, mammalian eyes.

### BACKGROUND

Solid pharmaceutically active implants that provide controlled release, for example, sustained release, of an active ingredient are able to provide a relatively uniform concentration of active ingredients in the body. Implants are particularly useful for providing a high local concentration at a particular target site for extended periods of time. Additionally, sustained release forms may reduce the number of doses of the drug required to be effective in treatment of a condition, and often reduce the occurrence of side effects and/or inconsistency in drug concentration found with traditional drug therapies.

However, many current formulations of sustained release implants have been found to have release profiles that do not provide relatively constant or consistent level of active component. For example, certain controlled release implants that are designed to provide consistent, sustained release, actually show little release until nearly complete erosion of the implant, at which time there is a dumping of the drug. Other preparations of sustained release implants are known to exhibit undesirable sigmoidal, or S-shaped, release profiles, wherein there is a clear inconsistency in the release rate of the drug over time.

It would be advantageous to provide eye implantable drug delivery systems, and methods of using such systems, having more consistent sustained release rates, delayed release rates, or other controlled and/or modified release rates for effective treatment of ocular diseases and disorders.

Macular degeneration, such as age related macular degeneration (“AMD”) is the leading cause of blindness in the world. It is estimated that thirteen million Americans have evidence of macular degeneration. Macular degeneration results in a break down the macula, the light-sensitive part of the retina responsible for the sharp, direct vision needed to read or drive. Central vision is especially affected. Macular degeneration is diagnosed as either dry (atrophic) or wet (exudative). The dry form of macular degeneration is more common than the wet form of macular degeneration, with about 90% of AMD patients being diagnosed with dry AMD. The wet form of the disease usually leads to more serious vision loss. Macular degeneration can produce a slow or sudden painless loss of vision. The cause of macular degeneration is not clear. The dry form of AMD may result from the aging and thinning of macular tissues, depositing of pigment in the macula, or a combination of the two processes. With wet AMD, new blood vessels grow beneath the retina and leak blood and fluid. This leakage causes retinal cells to die and creates blind spots in central vision. Current treatments for macular degeneration are generally limited to those aimed at preventing further progression of the disease. For example, laser photocoagulation is used to destroy blood vessels that have encroached on the macula.

Macular edema (“ME”) can result in a swelling of the macula. The edema is caused by fluid leaking from retinal blood vessels. Blood leaks out of the weak vessel walls into a very small area of the macula which is rich in cones, the nerve endings that detect color and from which daytime vision depends. Blurring then occurs in the middle or just to the side of the central visual field. Visual loss can progress over a period of months. Retinal blood vessel obstruction, eye inflammation, and age-related macular degeneration have all been associated with macular edema. The macula may also be affected by swelling following cataract extraction. Current treatment for ME includes topical anti-inflammatory drops. In some cases, medication is injected near the back of the eye for a more concentrated effect. Oral medications are also sometimes prescribed.

Glaucoma is a serious ocular condition characterized by increased ocular pressure and loss of retinal ganglion cells. Damage caused by glaucoma is thought to be irreversible. Current treatments for early stage glaucoma usually involve therapeutic eyedrops and oral medications used to lower ocular pressure.

Diabetic retinopathy is characterized by angiogenesis. Small blood vessels on the retina of the eye are damaged, resulting in the growth of abnormal blood vessels which proliferate and eventually leak and blur or otherwise obscure vision. Laser surgery is the current mainstay of treatment for diabetic retinopathy. Advanced proliferative diabetic retinopathy may be treated by vitrectomy, which includes removal of a portion of the vitreous and replacement with a clear replacement material. In any event, early treatment of diabetic retinopathy is essential to preventing permanent vision loss.

Uveitis involves inflammation of structures of the uvea. Treatment may consist of topical eyedrops or ointments containing cortiosteroids.

Retinitis pigmentosa is characterized by retinal degeneration. Retinitis pigmentosa is considered to be not one disease, but rather a group of diseases with common attributes. Visual problems common to retinitis pigmentosa include tunnel vision field, night blindness, glare problems, double vision and development of cataracts. Currently, there are no standard treatments available for retinitis pigmentosa, though it is believed that increasing intake of Vitamin A may slow progression of the disease.

Topically or orally administered medicinal agents, for example anti-inflammatory (i.e. immunosuppressive) agents, are currently a first line of treatment for many ocular conditions.

A major problem with topical and oral drug administration of drugs in treatment of the eye is the inability of the drug to achieve an adequate (i.e. therapeutic) intraocular concentration.

Systemic glucocorticoid administration is often used alone or in addition to topical glucocorticoids for the treatment of uveitis. However, prolonged exposure to high plasma concentrations (administration of 1 mg/kg/day for 2-3 weeks) of steroid is often necessary so that therapeutic levels can be achieved in the eye.

Unfortunately, these high drug plasma levels commonly lead to systemic side effects such as hypertension, hyperglycemia, increased susceptibility to infection, peptic ulcers, psychosis, and other complications. Cheng C. K. et al. “Intravitreal sustained-release dexamethasone device in the treatment of experimental uveitis”, Invest. Ophthalmol. Vis. Sci. 36:442-53 (1995); Schwartz, B. “The response of ocular pressure to corticosteroids”, Ophthalmol. Clin. North Am. 6:929-89 (1966); Skalka, H. W. et al. “Effect of corticosteroids on cataract formation”, Arch. Ophthalmol. 98:1773-7 (1980); and Renfro, L. et al. “Ocular effects of topical and systemic steroids”, Dermatologic Clinics 10:505-12 (1992).

Additionally, delivery to the eye of a therapeutic amount of an active agent can be difficult, if not impossible, for drugs with short plasma half-lives since the exposure of the drug to intraocular tissues is limited. A more efficient way of delivering a drug to treat an ocular condition is to place the drug directly in the eye.

Techniques such as intravitreal injection of a drug have shown promising results, but due to the short intraocular half-life of active agent, such as glucocorticoids (approximately 3 hours), intravitreal injections must be frequently repeated to maintain a therapeutic drug level. In turn, this repetitive process increases the potential for side effects such as retinal detachment, endophthalmitis, and cataracts. Maurice, D. M. “Micropharmaceutics of the eye”, Ocular Inflammation Ther. 1:97-102 (1983); Olsen, T. W. et al. “Human scleral permeability: effects of age, cryotherapy, transscleral diode laser, and surgical thinning”, Invest. Ophthalmol. Vis. Sci. 36:1893-1903 (1995); and Kwak, H. W. and D'Amico, D. J. “Evaluation of the retinal toxicity and pharmacokinetics of dexamethasone after intravitreal injection”, Arch. Ophthalmol. 110:259-66 (1992).

Additionally, topical, systemic, and periocular glucocorticoid treatment must be monitored closely due to toxicity and the long-term side effects associated with chronic systemic drug exposure sequelae. Rao, N. A. et al. (1997). “Intraocular inflammation and uveitis”, in: Basic and Clinical Science Course (San Francisco: American Academy of Ophthalmology, 1997-1998), Section 9, pp. 57-80, 102-103, 152-156; Schwartz, B. “The response of ocular pressure to corticosteroids”, Ophthalmol. Clin. North Am. 6:929-89 (1966).; Skalka, H. W. and Pichal, J. T. “Effect of corticosteroids on cataract formation” Arch. Ophthalmol. 98:1773-1777 (1980); Renfro, L. and Snow, J. S. “Ocular effects of topical and systemic steroids”, Dermatologic Clinics 10:505-12 (1992).; Bodor, N. et al. “A comparison of intraocular pressure elevating activity of loteprednoletabonate and dexamethasone in rabbits” Current Eye Research 11:525-30 (1992).

What is needed then are more effective systems and methods for treating ocular conditions. The present invention is concerned with and directed to implantable drug delivery systems and methods for treatment of these and other ocular conditions. The present systems and methods are useful for treating an anterior ocular condition, a posterior ocular condition, or an ocular condition which can be characterized as both an anterior ocular condition and a posterior ocular condition.

The following patents and additional publications include disclosure which is relevant to and/or helpful in understanding the present invention.

Weber et al., U.S. patent application Ser. No. 10/246,884, filed on Sep. 18, 2002, having Pub. No. US 2004/0054374 A1, describes apparatus and methods for delivering ocular implants into an eye of a patient.

Wong, U.S. Pat. No. 4,997,652 discloses biodegradable ocular implants, including encapsulated agents, and describes implanting microcapsules comprising hydrocortisone succinate into the posterior segment of the eye.

Wong, U.S. Pat. No. 5,164,188 discloses encapsulated agents for introduction into the suprachoroid of the eye, and describes placing microcapsules and plaques comprising hydrocortisone into the pars plana.

Wong et al., U.S. Pat. Nos. 5,443,505 and 5,766,242 disclose implants comprising active agents for introduction into a suprachoroidal space or an avascular region of the eye, and describes placing microcapsules and plaques comprising hydrocortisone into the pars plana.

Wong et al., U.S. Pat. No. 5,869,079 discloses combinations of hydrophilic and hydrophobic entities in a biodegradable sustained release implant, and describes a polylactic acid polyglycolic acid (PLGA) copolymer implant comprising dexamethasone.

Wong, U.S. Pat. No. 5,824,072 discloses implants for introduction into a suprachoroidal space or an avascular region of the eye, and describes a methylcellulose (i.e. non-biodegradable) implant comprising dexamethasone.

Zhou et al. discloses a multiple-drug implant comprising 5-fluorouridine, triamcinolone, and human recombinant tissue plasminogen activator for intraocular management of proliferative vitreoretinopathy. Zhou, T., et al. “Development of a multiple-drug delivery implant for intraocular management of proliferative vitreoretinopathy”, Journal of Controlled Release 55: pp. 281-295.

Heller, “Biodegradable Polymers in Controlled Drug Delivery”, in: CRC Critical Reviews in Therapeutic Drug Carrier Systems, Vol. 1, (CRC Press, Boca Raton, Fla., 1987), pp 39-90, describes encapsulation for controlled drug delivery. Heller, in: Hydrogels in Medicine and Pharmacy, N. A. Peppes ed., Vol. III, (CRC Press, Boca Raton, Fla., 1987), pp 137-149, describes bioerodible polymers.

Anderson et al., Contraception 13:375, (1976), and Miller et al., J. Biomed. Materials Res. 11:711, (1977) describe various properties of poly(dL-lactic acid).

Brine, U.S. Pat. No. 5,075,115 discloses controlled release formulations with lactic acid polymers and co-polymers.

Di Colo, Biomaterials 13:850-856 (1992) describes controlled drug release from hydrophobic polymers.

Olejnik, et al. U.S. Pat. No. 6,074,661 discloses an implantable device for treatment of an eye, wherein the device incorporates a retinoid for improving the biocompatibility of the device in eye tissue.

Wong, U.S. Pat. No. 6,699,493 discloses a method for reducing or preventing transplant rejection in the eye and intraocular implants for use therefore.

Other documents that are also relevant or otherwise helpful in understanding the present invention are U.S. patent application Ser. Nos. 09/693,008, filed on Jul. 5, 2000; 10/246,884, filed on Sep. 18, 2002; 10/327,018, filed on Dec. 20, 2002 and 10/340,237, filed on Jan. 9, 2003.

The entire disclosure of each of the documents cited hereinabove is incorporated herein in its entirety by this reference.

### SUMMARY

The present invention provides new drug delivery systems, and methods of using such systems, for modified, controlled release of a drug into an eye, for example, to achieve one or more desired therapeutic effects. The present systems and methods advantageously provide for desired or substantially predetermined drug release rates, such as for example, a desired or substantially predetermined burst effect of drug release into the eye. Thus, the patient in whose eye the present drug delivery system placed is benefited by having a controlled release of the active component within the eye for treatment of an ocular condition over a predetermined period of time. For example, in accordance with some embodiments of the invention, a patient with the present drug delivery system placed, for example, implanted within an eye has an initial burst effect of an active component, followed by a substantially consistent level of an active component available for consistent treatment of the eye over a relatively long period of time, for example, on the order of at least about 1 week or at least about 1 month or at least about 3 months or longer. Such initial controllable burst effect and consistent active component release rates facilitate obtaining successful treatment results.

Advantageously, the present delivery devices preferably are at least partially biodegradable so that removal of the device, after substantially complete active component release, is not required. The present drug delivery systems are relatively straightforward in structure, and can be relatively easily made and used to treat a wide variety of ocular conditions.

In one broad aspect of the invention, the drug delivery systems comprise one or more elements, hereinafter, sometimes interchangeably referred to as “implants,” sized and adapted for placement into an eye, for example, into a location of an eye such as one of an anterior chamber of an eye, a posterior chamber of an eye, a vitreous, cornea, scleral, retina, meningeal space, optic nerve, and/or intraoptic nerve of an eye. Such elements preferably include a therapeutic component, sometimes referred to elsewhere herein as an “active component” or a “therapeutically active component” comprising one or more active agents, and a matrix component comprising one or more substantially inactive components, for example, a polymeric matrix material.

In accordance with one aspect of the invention, the element, may have a controlled porosity that is effective in controlling a release rate of the therapeutic component from the element into the eye in which the element is placed. For example, the matrix component of the element may be structured to define regular or irregular pores or micropores, preferably disposed throughout the element.

Generally, the porosity of the element is selected to be effective in controlling, for example, shortening or extending, the release rate of the therapeutic component from the element relative to a similar or identical element without such a porosity, for example, relative to a similar or identical element that is relatively more solid or more densely structured throughout.

In accordance with another aspect of the invention, the element has a surface exhibiting a controlled roughness effective in controlling a release rate of the therapeutic component from the element. For example, the matrix component of the element may be structured to exhibit a roughened, for example, a substantially textured, surface.

Generally, the roughness of the surface of the element is effective in controlling a release rate profile, for example, including a burst effect of a release rate, of the therapeutic component.

The implant compositions, in accordance with the invention, can vary according to the ocular condition being treated, the preferred drug release profile, the particular active agent used, and the medical history of the patient.

At least a portion of the element preferably is biodegradable or bioerodible. For example, the matrix component is preferably biodegradable or bioerodable.

In the present context, a biodegradable or bioerodible material is one which degrades into physiologically acceptable degradation products under physiological conditions in the eye, or erodes into physiologically acceptable materials under physiological conditions in the eye.

In some embodiments of the present invention, the element advantageously comprises a controlled release implant including therapeutic component admixed with one or more matrix materials, for example, one or more polymeric materials, for example, one or more biodegradable or bioerodible polymeric materials. More specifically, the element may be structured, for example, may include a selected porosity and/or a roughening, effective in controlling a rate of release of the therapeutically active agents therefrom upon erosion or degradation of the inactive, bioerodible material.

The devices, systems and methods of the present invention can be used to deliver, in a controlled manner, any desired therapeutic agent, or combination of therapeutic agents, including an antibiotic agent, an antiviral agent, an antifungal agent, an anti-cancer agent, an antiglaucoma agent, an anti-inflammatory agent, an analgesic, an immunomodulatory agent, a macro-molecule, or a mixture thereof.

The systems of the invention may be structured such that the biodegradable polymer matrix may comprise at least about 10 percent, at least about 20 percent, at least about 30 percent, at least about 40 percent, at least about 50 percent, at least about 60 percent, at least about 70 percent, at least about 80 percent, at least about 90 percent of the element.

Therapeutic, active agents that may be used in the systems and methods of the present invention include, but are not limited to ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, dideoxyinosine, dideoxycytosine, dexamethasone, ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; .beta.-adrenergic blocker or .beta.-adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine, anihelminthic agents, e.g. ivermectin and suramin sodium; antiamebic agents, e.g. chloroquine and chlortetracycline; and antifungal agents, e.g. amphotericin, etc., anti-angiogenesis compounds such as anecortave acetate, retinoids such as Tazarotene, anti-glaucoma agents, such as brimonidine (Alphagan and Alphagan P), acetozolamide, bimatoprost (Lumigan), Timolol, mebefunolol; memantine; alpha-2 adrenergic receptor agonists; 2ME2; anti-neoplastics, such as vinblastine, vincristine, interferons; alpha., beta. and gamma., antimetabolites, such as folic acid analogs, purine analogs, and pyrimidine analogs; immunosuppressants such as azathiprine, cyclosporine and mizoribine; miotic agents, such as carbachol, mydriatic agents such as atropine, etc., protease inhibitors such as aprotinin, camostat, gabexate, vasodilators such as bradykinin, etc., and various growth factors, such epidermal growth factor, basic fibroblast growth factor, nerve growth factors, and the like.

An element or implant within the scope of the present invention can be formulated with particles of an active agent dispersed within a biodegradable polymer matrix. Release of the active agent can be achieved by erosion of the biodegradable polymer matrix and by diffusion of the particulate agent into an ocular fluid, for example, vitreal fluid, with contemporaneous or subsequent dissolution of the polymer matrix. By providing such element with a controlled porosity, as in some embodiments of the invention, release of the active agent is controlled based in part on a level of access of ocular fluid to the particulate agent through pores of the element.

In addition to the porosity and/or roughening of the implant as described elsewhere herein, the release kinetics of the implants of the present invention can be dependent in part on other factors, such as, for example, the surface area of the implant. A larger surface area exposes more of the implant composition to ocular fluid, causing faster erosion of the polymer matrix and faster dissolution of the active agent particles in the fluid. Therefore, the size and shape of the implant may also be used to control the rate of release, period of treatment, and active agent concentration at the site of implantation. At equal active agent loads, larger implants will deliver a proportionately larger dose, but depending on the surface to mass ratio, may possess a slower release rate.

Other factors which influence the release kinetics of active agent from the implant can include such characteristics as the size and shape of the implant, the size of the active agent particles, the solubility of the active agent, the ratio of active agent to polymer(s), the method of manufacture, the surface area exposed, and the erosion rate of the polymer(s). The release kinetics achieved by degradation or erosion of the element are different than that achieved through formulations which release active agents through polymer swelling, such as with crosslinked hydrogels. In that case, the active agent is not released through polymer erosion, but through polymer swelling and drug diffusion, which releases agent as liquid diffuses through the pathways exposed.

It is noted that the release rate of the active agent from systems in accordance with the invention can in some embodiments depend at least in part on the mechanism of degradation of the polymeric component or components making up the biodegradable polymer matrix. For example, condensation polymers may be degraded by hydrolysis (among other mechanisms) and therefore any change in the composition of the implant that enhances water uptake by the implant will likely increase the rate of hydrolysis, thereby increasing the rate of polymer degradation and erosion, and thus increasing the rate of active agent release.

The implants in accordance with the present invention may be of any geometry including particles, sheets, patches, plaques, films, discs, fibers, rods, and the like, or may be of any size or shape compatible with the selected site of implantation, as long as the implants have the desired release kinetics and deliver an amount of active agent that is therapeutic for the intended medical condition of the eye. The upper limit for the implant size will be determined by factors such as the desired release kinetics, toleration for the implant at the site of implantation, size limitations on insertion, and ease of handling. For example, the vitreous chamber is able to accommodate relatively large rod-shaped implants, generally having diameters of about 0.05 mm to 3 mm and a length of about 0.5 to about 10 mm. In one variation, the rods have diameters of about 0.1 mm to about 1 mm. In another variation, the rods have diameters of about 0.3 mm to about 0.75 mm. In yet a further variation, other implants having variable geometries but approximately similar volumes may also be used.

In some embodiments of the invention, the element is structured such that upon being placed, for example, implanted into an eye, for example into a vitreous of an eye, each exposed or outer surface of the element biodegrades or bioerodes at a substantially uniform rate and in a substantially uniform manner in relation to each other exposed or outer surface. Thus, in some embodiments of the invention, the element is structured to degrade or erode in the ocular environment at a rate and in a manner such that the configuration or shape of the element remains substantially consistent throughout the treatment period.

In accordance with the present invention, the elements may have predefined pores that are formed in the element due to preset extrusion parameters during manufacture of the element, or by other suitable means.

Similarly, a roughened surface on an element in accordance with some embodiments of the present invention may be formed by appropriate selection of extrusion parameters that will effectively provide a desired surface texture of the element.

The systems of the invention may comprise a plurality of the elements as described and shown herein.

The present invention also provides methods of treating an eye, for example including the step of placing a drug delivery system described herein into an eye.

Each and every feature described herein, and each and every combination of two or more of such features, is included within the scope of the present invention provided that the features included in such a combination are not mutually inconsistent.

Additional aspects and advantages of the present invention are set forth in the following description and claims, particularly when considered in conjunction with the accompanying drawings in which like parts bear like reference numerals.

### DRAWINGS

FIG. 1 shows a scanning electron microscope (SEM) image of a drug delivery system in accordance with an embodiment of the invention in which the system comprises an element or implant having a controlled porosity.

FIG. 2 shows a simplified perspective view of a drug delivery system in accordance with another embodiment of the invention in which the system comprises an element or implant having a controlled roughness.

FIG. 3 shows percentage of drug release on day 1 as function of average surface roughness (Ra), of drug delivery systems in accordance with the present invention.

FIG. 4 shows percentage of drug release on day 7 as function of average surface roughness (Ra) of drug delivery systems in accordance with the present invention.

FIG. 5 shows percentage of drug release on day 1 as function of root mean square (rms) average roughness (Rq) of drug delivery systems in accordance with the present invention.

FIG. 6 shows percentage of drug release on day 7 as function of root mean square (rms) average roughness (Rq) of drug delivery systems in accordance with the present invention.

FIG. 7 shows a percentage of drug release as a function of time for two samples of drug delivery systems of the invention, each line representing samples having a particular Ra value.

FIG. 8 shows a cross-sectional view of an eye.

### DESCRIPTION

The present drug delivery systems of the present invention are generally directed to controlled release drug delivery system implants and methods for the treatment of ocular conditions, such as an anterior ocular condition, a posterior ocular condition, or an ocular condition which can be characterized as both an anterior ocular condition and a posterior ocular condition.

As used herein, and as generally understood by those of skill in the art, an ocular condition can include a disease, aliment or condition which affects or involves the eye or one of the parts or regions of the eye. Broadly speaking, the eye includes the eyeball and the tissues and fluids which constitute the eyeball, the periocular muscles (such as the oblique and rectus muscles) and the portion of the optic nerve which is within or adjacent to the eyeball.

An anterior ocular condition generally refers to a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves, the conjunctiva, the cornea, the conjunctiva, the anterior chamber, the iris, the posterior chamber (behind the retina but in front of the posterior wall of the lens capsule), the lens or the lens capsule and blood vessels and nerve which vascularize or innervate an anterior ocular region or site. An anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye.

A posterior ocular condition generally refers to a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

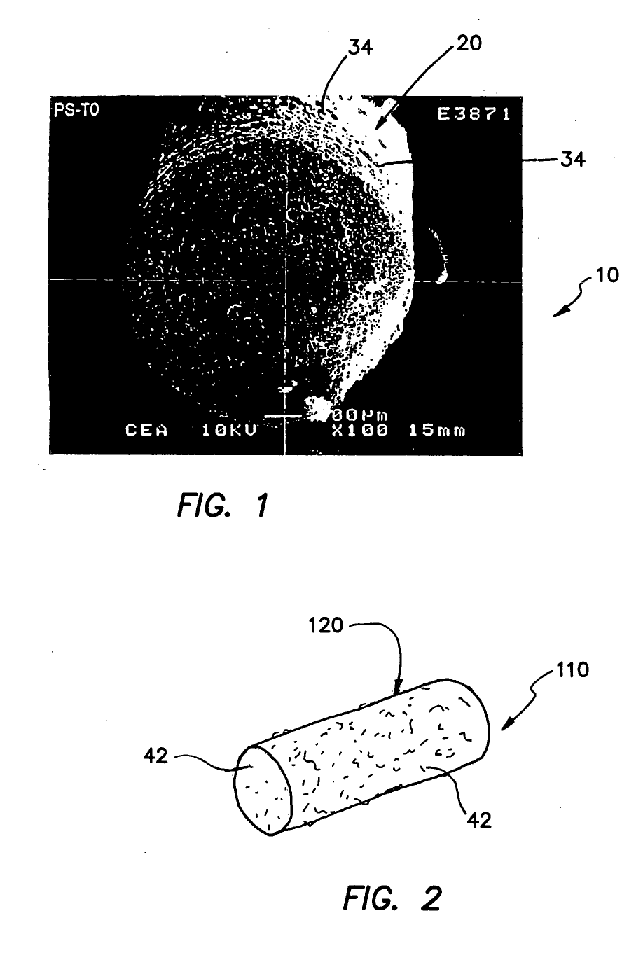

Referring now to FIG. 1, a drug delivery system in accordance with the present invention is shown generally at 10.

The system 10 generally comprises an element 20 sized and adapted for placement into an eye, such as the eye 300 shown in FIG. 8, said element 20 including a therapeutic component and a matrix component, the therapeutic component being located in combination with the matrix component, for example, the therapeutic component may be substantially uniformly distributed throughout the matrix component.

Advantageously, in accordance with one aspect of the invention, the element 20, for example, the matrix component thereof, has at least one of a controlled porosity and a controlled roughness, effective in controlling a release rate of the therapeutic component from the element 20 into an eye in which the element is placed.

For example, in accordance with one aspect of the invention, the element 20 includes a porosity selected to be effective in controlling the release rate of the therapeutic component from the element 20. For example, the system 10 may be structured such that an increase in porosity of the element 20 is effective in increasing the release rate of the therapeutic component into an eye in which the element 20 is placed.

FIG. 1 shows that, in this particular embodiment of the invention, the element 20 has a porosity defined by pores 34 of substantially irregular size and shape. The pores 34 preferably are disposed throughout the element 20, for example, the element 20 may have openings or orifices defined within an exterior surface of the element 20 as well as a porous interior defined by open cavities and/or channels, for example irregular cavities and/or channels.

Alternatively, in other embodiments of the invention, the element 20 may have a porous outer surface portion having a defined or limited depth, and a substantially solid, substantially non-porous interior portion. In such a case, the element may be at least partially biodegradable, and controlled release of the drug from the element may be exhibited by a rapid initial release of therapeutic agent during erosion of the porous outer surface portion, followed by a slower, less concentrated, more sustained release of the therapeutic agent from the relatively more solid or non-porous interior portion.

Generally, it has been discovered that an increase in porosity, for example, an increase in pore size and/or quantity of pores, leads to an increase in a drug release rate from the element. Thus, the present systems can be tailored to meet the desired treatment goals by appropriate selection of element porosity.

Although not wishing to be bound by any particular theory of operation, it is believed that pores 34 within the element, for example, apertures, channels, recesses, and the like, provide the element 20 with an increased exposed surface for contact with the ocular environment, relative to an identical element without such pores, thereby facilitating or enhancing a rate of release of the active agent from the element 20. Generally, a relatively small pore size, for example, micropore size, may contribute to a relatively slower rate of diffusion and interchange of ocular fluid and therapeutic agent within the ocular site containing the element, thus extending the time that the drug is available to the eye and decreasing the release rate of the drug. Likewise, relatively larger pore size may contribute to more rapid diffusion and interchange of ocular fluid, thus decreasing the time that the drug is available to the eye and increasing the release rate of the drug. The relative number of pores and spacing between pores in the elements may be modified to provide further control of the release rate. As used herein, the term “porous” refers to a property of the element that is defined by holes, pores or channels hereinafter generally referred to as “pores”, that allow diffusion or permeation of fluids between the element and the ocular environment, for example, pores may have a diameter ranging in size from about 0.2 micron to about 300 microns, or greater. As used herein, “microporous” refers more specifically to pores that are typically less than about 0.2 microns. Such pores are more clearly visible using a scanning electron microscope equipment.

Other parameters which generally affect the release kinetics from the element 20 include the size of the therapeutic component or drug particles entrapped in the element 20, water solubility of the therapeutic component or drug, the ratio of therapeutic component or drug to polymer, and the erosion rate of the polymer present in the element 20.

It is to be appreciated that shape of the element 20 is a general consideration in formulation of an element having a desired release profile. Thus, although the system 10 shown in FIG. 1 comprises element 20 having a substantially cylindrical form with circular cross-section perpendicular to a longitudinal axis of the element, it is to be appreciated that other elements having shapes with cross-sections other than circular, for example triangular, rectangular, elliptical cross-sections, are also included within the scope of the present invention. Irregular shapes may also be used.

Suitable polymeric materials or compositions for use in the systems of the present invention include those materials which are compatible, that is biocompatible, with the eye so as to cause no substantial interference with the functioning or physiology of the eye.

The matrix component may comprise materials which are at least partially, for example, are substantially completely, biodegradable or bioerodible (these terms are generally used interchangeably herein), when exposed to the ocular environment. As the matrix material degrades within the eye, the therapeutic component is released into the eye, providing substantially consistent, for example, substantially constant therapeutic benefit thereto.

In other embodiments of the invention, the matrix component is made of materials that are not biodegradable, or are not substantially biodegradable, when exposed to the ocular environment. In this case, the element is structured to allow diffusion of ocular fluid and the therapeutic component through the pores of the element.

The selection of the matrix component material, for example, polymeric material, used in the present systems can vary with the desired release kinetics, patient tolerance, the nature of the disease to be treated, and the like.

Biodegradable polymers which can be used include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked.

For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen can be present as amide, cyano, and amino. An exemplary list of biodegradable polymers that can be used are described in Heller, “Biodegradable Polymers in Controlled Drug Delivery”, in: CRC Critical Reviews in Therapeutic Drug Carrier Systems, Vol.1. (CRC Press, Boca Raton, Fla., 1987).

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers.

Biodegradable polymer matrices that include mixtures of hydrophilic and hydrophobic ended PLGA may also be employed, and are useful in modulating polymer matrix degradation rates. Hydrophobic ended (also referred to as capped or end-capped) PLGA has an ester linkage hydrophobic in nature at the polymer terminus. Typical hydrophobic end groups include, but are not limited to alkyl esters and aromatic esters. Hydrophilic ended (also referred to as uncapped) PLGA has an end group hydrophilic in nature at the polymer terminus. PLGA with a hydrophilic end groups at the polymer terminus degrades faster than hydrophobic ended PLGA because it takes up water and undergoes hydrolysis at a faster rate (Tracy et al., Biomaterials 20:1057-1062 (1999)). Examples of suitable hydrophilic end groups that may be incorporated to enhance hydrolysis include, but are not limited to, carboxyl, hydroxyl, and polyethylene glycol. The specific end group will typically result from the initiator employed in the polymerization process. For example, if the initiator is water or carboxylic acid, the resulting end groups will be carboxyl and hydroxyl. Similarly, if the initiator is a monofunctional alcohol, the resulting end groups will be ester or hydroxyl.

The composition of the implants may be monolithic, that is, having the therapeutic component substantially uniformly distributed throughout the matrix component, for example, throughout the polymeric material present in the implant, or the implants may have encapsulated reservoirs for example, particles and/or other relatively concentrated forms, of therapeutic component interspersed throughout the implant, for example, throughout the polymeric material in the implant.

Among the useful polysaccharides are, without limitation, calcium alginate, and functionalized celluloses, particularly carboxymethylecellulose esters characterized by being water insoluble, a molecular weight of about 5 kD to 500 kD, etc.

Other polymers of interest include, without limitation, polyvinyl alcohol, polyesters, polyethers and combinations thereof which are biocompatible and may or may not be biodegradable and/or bioerodible.

Some preferred characteristics of the polymers or polymeric materials for use in the present invention may include biocompatibility, compatibility with the therapeutic component, ease of use of the polymer in making the drug delivery systems of the present invention, a halflife in the physiological environment of at least about 6 hours, preferably greater than about one day, not significantly increasing the viscosity of the vitreous, and water insolubility.

The biodegradable polymeric materials are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, where the polymers may be employed as varying layers or mixed.

Alternatively or additionally, various non-biodegradable polymeric compositions may be employed in the implants. The non-biodegradable polymeric composition employed may allow for release of the drug by, for example, solution/diffusion or leaching mechanisms. The non-biodegradable polymeric compositions employed may be varied according to the compatibility of the polymer with the drug or other active agent to be employed, ease of manufacture, the desired rate of release of the drug, desired density or porosity, and the like. Various non-biodegradable polymers which may be employed are described in U.S. Pat. Nos. 4,303,637; 4,304,765; 4,190,642; 4,186,184; 4,057,619; 4,052,505; 4,281,654; 4,959,217; 4,014,335; 4,668,506; 4,144,317. The non-biodegradable polymers may be homopolymers, copolymers, straight, branched-chain, or cross-linked derivatives.

Exemplary biocompatible, non-biodegradable polymers of particular interest include polycarbamates or polyureas, particularly polyurethanes, polymers which may be cross-linked to produce non-biodegradable polymers such as cross-linked poly(vinyl acetate) and the like. Also of particular interest are ethylene-vinyl ester copolymers having an ester content of 4 to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, Ethylene-vinyl ester copolymers including ethylene-vinyl acetate copolymers for the manufacture of diffusional ocular drug delivery devices where the drug dissolves in and passes through the polymer by diffusion are described in U.S. Pat. Nos. 4,052,505 and 4,144,317.

Additional exemplary naturally occurring or synthetic non-biodegradable polymeric materials include poly(methylmethacrylate), poly(butylmethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(-vinylidene chloride), poly(acrylonitrile), cross-linked poly(vinylpyrrolidone), poly(trifluorochloroethylene), chlorinated poly(ethylene), poly(4,4′-isopropylidene diphenylene carbonate), vinylidene chloride-acrylonitrile copolymer, vinyl chloridediethyl fumarate copolymer, silicone, silicone rubbers (especially the medical grade), poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls), poly(acrylate), poly(methacrylate), poly(oxides), poly(esters), poly(amides), and poly(carbonates).

Biodegradable or non-biodegradable hydrogels may also be employed in the implants of the subject invention. Hydrogels are typically a copolymer material, characterized by the ability to imbibe a liquid. Exemplary non-biodegradable hydrogels which may be employed and methods of making these hydrogels are described in U.S. Pat. Nos. 4,959,217 and 4,668,506, the entire disclosures of which are incorporated herein by reference.

In addition to the controlled porosity and/or roughening of the implant, in some embodiments of the invention which employ a non-biodegradable polymer, the rate of release of the drug will be solution/diffusion controlled. The rate of diffusion of drug through the non-biodegradable polymer may be affected by drug solubility, polymer hydrophilicity, extent of polymer cross-linking, expansion of the polymer upon water absorption so as to make the polymer more permeable to the drug, and the like.

The element 20 advantageously is structured to have a lifetime at least equal to the desired period of therapeutic component administration in the eye, and may have lifetimes of about 5 to about 10 times the desired period of administration. The period of administration may be at least about 3 days, at least about 7 days, at least about 15 days, at least about 20 days, at least about 30 days or longer.

The therapeutic component useful in the present invention may include any suitable pharmacologically active agent or therapeutic agent for which sustained, modified, extended, delayed, or otherwise controlled release in the eye, is desirable. Advantageously, the therapeutic component is preferably sufficiently soluble in the vitreous of the eye such that it will be present at a pharmacologically or otherwise therapeutically effective dose. Pharmacologic or therapeutic agents which may find use in the present systems, include, without limitation, those disclosed in U.S. Pat. Nos. 4,474,451, columns 4-6 and 4,327,725, columns 7-8, which disclosures are incorporated herein by reference.

Pharmacological or therapeutic agents of interest include hydrocortisone (5-20 mcg/l as plasma level), gentamycin (6-10 mcg/ml in serum), 5-fluorouracil (about 0.30 mg/kg body weight in serum), sorbinil, IL-2, TNF, Phakan-a (a component of glutathione), thioloa-thiopronin, Bendazac, acetylsalicylic acid, trifluorothymidine, interferon (alpha., beta. and gamma.), immune modulators, e.g. lymphokines, monokines, and growth factors, etc.

Pharmacological or therapeutic agents of particular interest include, without limitation, anti-glaucoma drugs, such as the beta-blockers, such as timolol maleate, betaxolol and metipranolol; mitotics, such as pilocarpine, acetylcholine chloride, isofluorophate, demacarium bromide, echothiophate iodide, phospholine iodide, carbachol, and physostigimine; epinephrine and salts, such as dipivefrin hydrochloride; and dichlorphenamide, acetazolamide and methazolamide; anti-cataract and anti-diabetic retinopathy drugs, such as aldose reductase inhibitors, such as tolrestat, lisinopril, enalapril, and statil; thiol cross-linking drugs other than those considered previously; anti-cancer drugs, such as retinoic acid, methotrexate, adriamycin, bleomycin, triamcinoline, mitomycin, cis-platinum, vincristine, vinblastine, actinomycin-D, ara-c, bisantrene, CCNU, activated cytoxan, DTIC, HMM, melphalan, mithramycin, procarbazine, VM26, VP16, and tamoxifen; immune modulators, other than those indicated previously; anti-clotting agents, such as tissue plasminogen activator, urokinase, and streptokinase; anti-tissue damage agents, such as superoxide dismutase; proteins and nucleic acids, such as mono- and polyclonal antibodies, enyzmes, protein hormones and genes, gene fragments and plasmids; steriods, particularly anti-inflammatory or anti-fibrous drugs, such as cortisone, hydrocortisone, prednisolone, prednisome, dexamethasone, peogesterone-like compounds, medrysone (HMS) and fluorometholone; non-steroidal anti-inflammatory drugs, such as ketrolac tromethamine, dichlofenac sodium and suprofen; antibiotics, such as loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cepholothin, colistin, erythromycin, streptomycin, neomycin, sulfacetamide, vancomycin, silver nitrate, sulfisoxazole diolamine, and tetracycline; other antipathogens, including anti-viral agents, such as idoxuridine, trifluorouridine, vidarabine (adenine arabinoside), acyclovir (acycloguanosine), pyrimethamine, trisulfapyrimidine-2, clindamycin, nystatin, flucytosine, natamycin, miconazole and piperazie derivatives, e.g. diethylcarbamazine; cycloplegic and mydriatic agents, such as atropine, cyclogel, scopolamine, homatropine and mydriacyl; and the like and mixtures thereof.

Other agents useful in the systems of the present invention include, without limitation, anticholinergics, anticoagulants, antifibrinolytic agents, antihistamines, antimalarials, antitoxins, chelating agents, hormones, immunosuppressives, thrombolytic agents, vitamins, salts, desensitizing agents, prostaglandins, amino acids, metabolites, antiallergenics, and the like and mixtures thereof.

In one embodiment of the invention, the active agent is methotrexate. In another embodiment, the active agent is a retinoic acid. In another embodiment, the active agent is an anti-inflammatory agent such as a nonsteroidal anti-inflammatory agent. Nonsteroidal anti-inflammatory agents that may be used include, but are not limited to, aspirin, diclofenac, flurbiprofen, ibuprofen, ketorolac, naproxen, and suprofen. In a further variation, the anti-inflammatory agent is a steroidal anti-inflammatory agent.

The steroidal anti-inflammatory agents that may be used in the systems of the present invention include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

In one aspect of the invention, cortisone, dexamethasone, fluocinolone, hydrocortisone, methylprednisolone, prednisolone, prednisone, and triamcinolone, and their derivatives, are preferred steroidal anti-inflammatory agents. In another aspect of the invention, the steroidal anti-inflammatory agent is dexamethasone. In another aspect of the invention, the biodegradable implant includes a combination of two or more steroidal anti-inflammatory agents.

The active agent, such as a steroidal anti-inflammatory agent, can comprise from about 10% to about 90% by weight of the element or implant. In one variation, the agent is from about 40% to about 80% by weight of the implant. In a preferred variation, the agent comprises about 60% by weight of the implant. In a more preferred embodiment of the present invention, the agent can comprise about 50% by weight of the implant.

Other agents may be employed in the formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The implants in accordance with the present invention can also include hydrophilic or hydrophobic compounds that accelerate or retard release of the active agent. Additionally, release modulators such as those described in U.S. Pat. No. 5,869,079 can be included in the implants. The amount of release modulator employed will be dependent on the desired release profile, the activity of the modulator, and on the release profile of the glucocorticoid in the absence of modulator. Where the buffering agent or release enhancer or modulator is hydrophilic, it may also act as a release accelerator. Hydrophilic additives act to increase the release rates through faster dissolution of the material surrounding the drug particles, which increases the surface area of the drug exposed, thereby increasing the rate of drug diffusion. Similarly, a hydrophobic buffering agent or enhancer or modulator can dissolve more slowly, slowing the exposure of drug particles, and thereby slowing the rate of drug diffusion.

In a particularly advantageous embodiment of the invention, the systems suitable for treating inflammation-mediated conditions of the eye are provided. The term “inflammation-mediated condition of the eye” is meant to include any condition of the eye which may benefit from treatment with an anti-inflammatory agent, and is meant to include, but is not limited to, uveitis, macular edema, acute macular degeneration, retinal detachment, ocular tumors, fungal or viral infections, multifoca1 choroiditis, diabetic uveitis, proliferative vitreoretinopathy (PVR), sympathetic opthalmia, Vogt Koyanagi-Harada (VKH) syndrome, histoplasmosis, and uveal diffusion.

For example, the systems may comprise an element, such as element 20, structured for being implanted into the vitreous of the eye wherein the therapeutic component comprises a steroidal anti-inflammatory agent, for example but not limited to, dexamethasone, and a bioerodible polymeric material, for example a polylactic acid/polyglycolic acid copolymer. The element 20 preferably delivers the agent to the vitreous in an amount sufficient to reach a concentration equivalent to at least about 0.05 μg/ml dexamethasone within about 48 hours and maintains a concentration equivalent to at least about 0.03 μg/ml dexamethasone for at least about three weeks. In another embodiment of the invention, the element 20 preferably delivers the agent to the vitreous in an amount sufficient to reach a concentration equivalent to at least about 0.2 μg/ml dexamethasone within about 6 hours and maintains a concentration equivalent to at least about 0.01 pg/ml dexamethasone for at least about three weeks.

“A concentration equivalent to dexamethasone”, as used herein, refers to the concentration of a steroidal anti-inflammatory agent necessary to have approximately the same efficacy in vivo as a particular dose of dexamethasone. For example, hydrocortisone is approximately twentyfivefold less potent than dexamethasone, and thus a 25 mg dose of hydrocortisone would be equivalent to a 1 mg dose of dexamethasone. One of ordinary skill in the art would be able to determine the concentration equivalent to dexamethasone for a particular steroidal anti-inflammatory agent from one of several standard tests known in the art. Relative potencies of selected corticosteroids may be found, for example, in Gilman, A. G., et al., eds. (1990). Goodman and Gilman's: The Pharmacological Basis of Therapeutics. 8th Edition, Pergamon Press: New York, p. 1447, which is incorporated herein by this specific reference.

In other embodiments, the implant or element 20 delivers the agent to the vitreous in an amount sufficient to reach a concentration equivalent to at least about 0.3 μg/ml, or at least about 0.5 μg/ml, or at least about 0.75 μg/ml, or at least about 1.0 μg/ml, or at least about 2.0 μg/ml dexamethasone within about 4 hours, or within about 6 hours, or within about 8 hours, or within about 10 hours, or within about 24 hours.

A concentration equivalent to at least about 0.01 μg/ml, or at least about 0.02 μg/ml, or at least about 0.03 μg/ml, or at least about 0.05 μg/ml, or at least about 0.07 μg/ml dexamethasone may be maintained for an extended period of time (e.g., at least about three weeks or longer). The preferred concentration levels of therapeutic component or drug in the vitreous may vary according to the inflammatory mediated condition being treated. For example, for treating uveitis, a concentration equivalent of at least about 0.01 to 0.1 μg/ml dexamethasone is preferred.

In one embodiment, the concentration or therapeutic component is maintained for least about four weeks. In other embodiments, the concentration is maintained for at least about five weeks, or at least about six weeks, or at least about seven weeks, or at least about eight weeks, or at least about nine weeks, or at least about 10 weeks, or at least about 12 weeks or longer. The preferred duration of therapeutic component or drug release may be determined by the inflammatory mediated condition being treated. For treating uveitis, a drug release duration of at least about three weeks is preferable, more preferably at least about four weeks. In one embodiment, more than one implant or element 20 may be sequentially implanted into the vitreous in order to maintain therapeutic component or drug concentrations for even longer periods.

In some embodiments of the present invention, the controlled porosity and/or the controlled roughness is effective in releasing between about 1% to about 25%, about 5% to about 20%, or about 15% of the therapeutic component from the element within about one day of the element being placed in an eye.

In other embodiments of the present invention, the controlled porosity and/or the controlled roughness is effective in releasing between about 1% to about 25%, about 5% to about 20%, or about 15% of the therapeutic component from the element within about seven days to about 14 days of the element being placed in an eye.

The implants or elements 20 of the present invention may be inserted into the eye, for example the vitreous chamber of the eye, by a variety of methods, including placement by forceps or by trocar following making a 2-3 mm incision in the sclera. The method of placement may influence the therapeutic component or drug release kinetics. For example, implanting the element 20 with a trocar may result in placement of the element 20 deeper within the vitreous than placement by forceps, which may result in the implant being closer to the edge of the vitreous. The location of the placed or implanted element 20 may influence the concentration gradients of therapeutic component or drug surrounding the element, and thus influence the release rates (e.g., an element placed closer to the edge of the vitreous will result in a slower release rate).

The formulation of the implants in accordance with the present invention may vary according to the desired therapeutic component release profile, the particular therapeutic component used, the condition being treated, and the medical history of the patient.

In some embodiments of the invention, the element 20 is formulated with particles of a steroidal anti-inflammatory agent entrapped within a bioerodible polymer matrix, for example a polylactic acid polyglycolic acid (PLGA) copolymer. After implantation of the element 20 in the eye, release of the agent into the eye is achieved by erosion of element 20 at the exposed surface of the element 20 as well as within the element due to contact of ocular fluid with an interior of the element based on the nature and degree of porosity of the element.

Preferably, the steroidal anti-inflammatory agent is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, fonnocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide and the like and mixtures thereof. In a preferred embodiment, the steroidal anti-inflammatory agent is selected from the group consisting of cortisone, dexamethasone, hydrocortisone, methylprednisolone, prednisolone, prednisone, triamcinolone and the like and mixtures thereof. In a more preferred embodiment, the steroidal anti-inflammatory agent is dexamethasone. In another embodiment, the bioerodible implant comprises more than one steroidal anti-inflammatory agent.

The amount or concentrations of therapeutic component employed in the element 20 will vary depending on the effective dosage required and rate of release.

For embodiments of the invention employing steroidal anti-inflammatory agents, the polymers may comprise, for example, polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate.

Copolymers of glycolic and lactic acid are of particular interest, where the rate of biodegradation is controlled by the ratio of glycolic to lactic acid. The % of polylactic acid in the polylactic acid polyglycolic acid (PLGA) copolymer can be 0-100%, preferably about 15-85%, more preferably about 35-65%. In a particularly preferred embodiment, a 50/50 PLGA copolymer is used. The most rapidly degraded copolymer has roughly equal amounts of glycolic and lactic acid, where either homopolymer is more resistant to degradation. The ratio of glycolic acid to lactic acid will also affect the brittleness of in the element, where a more flexible element is desirable for larger geometries.

Other agents may be employed in the element 20 for a variety of purposes. In addition to the therapeutic component, effective amounts of buffering agents, preservatives and the like may be employed. Suitable water soluble preservatives include sodium bisulfite, sodium thiosulfate, ascorbate, benzalkonium chloride, chlorobutanol, thimerosal, phenylmercuric borate, parabens, benzyl alcohol, phenylethanol and the like and mixtures thereof. These agents may be present in amounts of from 0.001 to about 5% by weight and preferably 0.01 to about 2% by weight. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such, the buffering agent may be as much as about 5% by weight of the total implant or element 20.

Turning now to FIG. 2, another aspect of the invention is shown. More specifically, the present invention further provides a drug delivery system 110 sized and adapted for placement into an eye, such as the eye 300 shown in FIG. 8.

Except as expressly described herein, system 110 is similar to system 10 and features of system 110 which correspond to features of system 10 are designated by the corresponding reference numerals increased by 100.

The drug delivery system 110 generally comprises an element 120 including a therapeutic component and a matrix component, the therapeutic component being located in the matrix component, for example, substantially uniformly distributed throughout the matrix component. Advantageously, in accordance with this aspect of the invention, at least a portion of the element 120 includes an outer surface 42 having a controlled roughness, or a selected degree of roughness, effective in controlling a release rate of the therapeutic component from the element 120.

Advantageously, in some embodiments of the invention, the roughness of outer surface 42 is be selected to be effective in controlling a burst effect of a release rate of the therapeutic component from the element 120.

In accordance with the present invention, it has been discovered that as surface roughness is increased, the concentration of drug initially released through the roughened surface, after implantation of the element 120 in the eye, is also increased.

A USP approved method for dissolution or release test can be used to measure the rate of release (USP 23; NF 18 (1995) pp. 1790-1798). For example, using the infinite sink method, a weighed sample of the drug delivery system 110 is added to a measured volume of a solution containing 0.9% NaCl in water, where the solution volume will be such that the drug concentration is after release is less than 20%, and preferably less than 5%, of saturation. The mixture is maintained at 37° C. and stirred slowly to ensure drug diffusion after bioerosion. The appearance of the dissolved drug as a function of time may be followed by various methods known in the art, such as spectrophotometrically, HPLC, mass spectroscopy, etc.

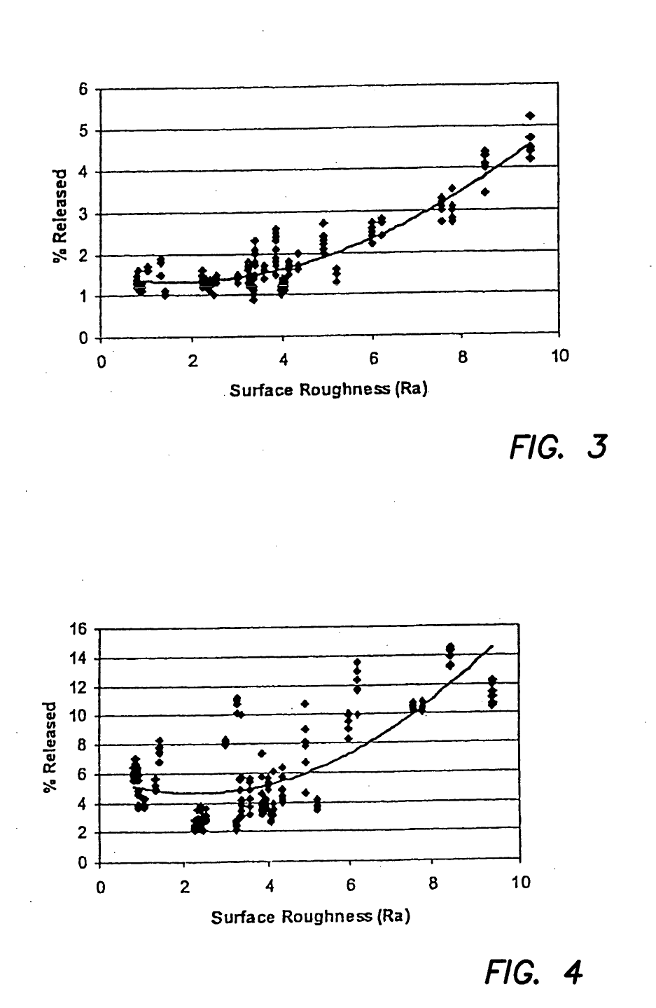

FIGS. 3 and 4 are graphs showing percentage of drug release from different element samples as a function of surface roughness (Ra) on Day 1 and Day 7, respectively.

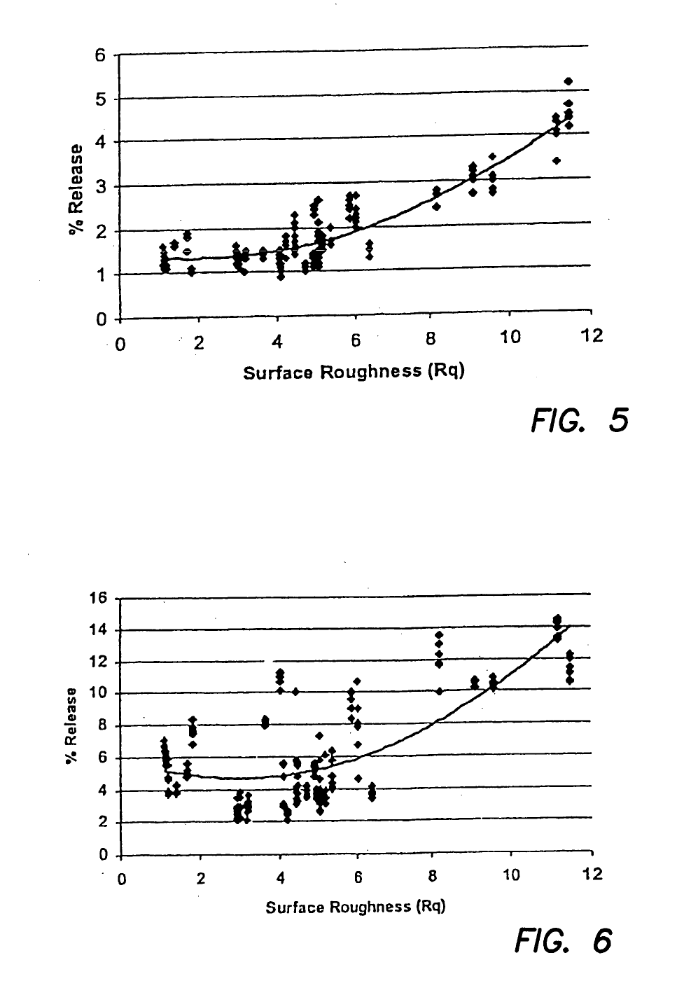

FIGS. 5 and 6 are similar graphs showing percentage of drug release from element samples as a function of surface roughness (Rq), on Day 1 and Day 7, respectively.

Roughness values Ra and Rq each generally represent a quantifiable, measurable value, indicated by a numerical value. Ra, also known as the arithmetic average, represents an average roughness. This value can be calculated by the area between the roughness profile and a mean line, or the integral of the absolute value of the roughness profile height over the evaluation line. Graphically, the average roughness is the area between the roughness profile and its center line divided by the evaluation length.

$${R}_{a} = \frac{1}{L} {\int }_{0}^{L} \vert r ( x ) \vert ⅆ x$$

Rq is the root mean square (rms) average roughness of a surface. It is calculated from another integral of roughness of the drug delivery device.

$${R}_{q} = \sqrt{\frac{1}{L} {\int }_{0}^{L} {r}^{2} ( x ) ⅆ x}$$

It is noted that persons of ordinary skill in the art understand and are aware of these and other means for calculating and/or otherwise determining roughness surface values and the present invention is not limited to any particular means of determining roughness surface values.

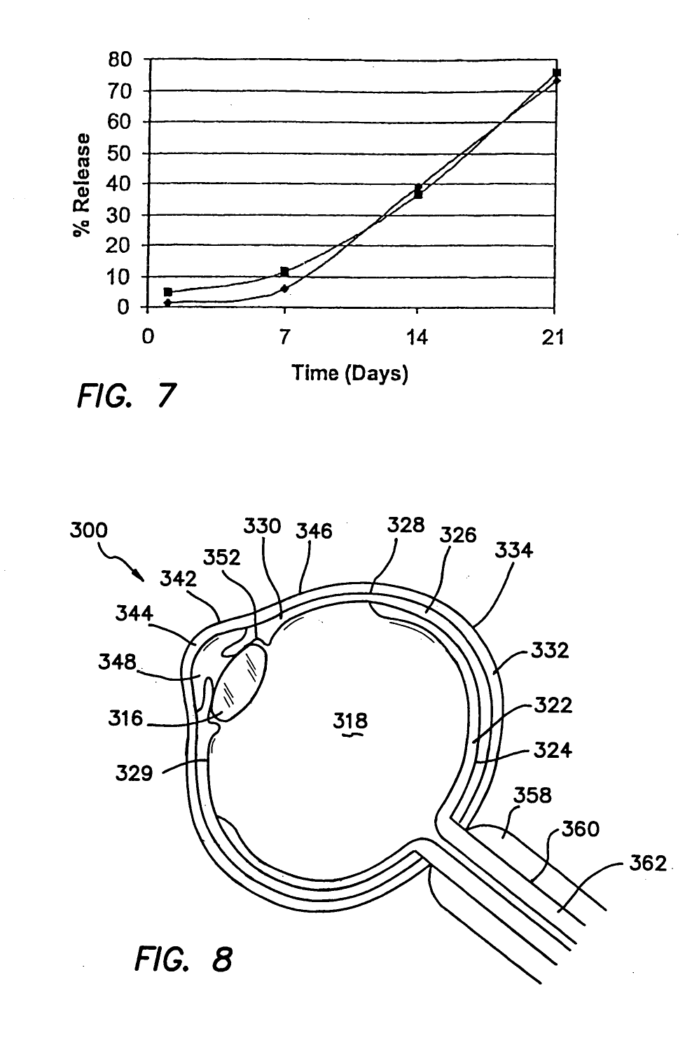

FIG. 7 shows a percentage of drug release as a function of time for two different lots (Sample 1, and Sample 2) of drug delivery elements in accordance with the present invention. Sample 1 has an Ra value of about 0.875. Sample 2 has an Ra value of about 9.427. Day 1 and day 7 average percent drug release are about 1.2% and about 6.1% respectively, for Sample 1. Day 1 and day 7 average percent drug release are about 4.6% and 11.3% respectively, for Sample 2.

It has been discovered that for biodegradable implants in accordance with the present invention, as surface roughness increases, the percentage of drug released from the elements also increases. There is a strong correlation between roughness values (Ra) and (Rq) and percentage of drug released on Day 1 (FIGS. 3 and 5). It can also be determined that there is a weaker correlation between roughness values and percentage of drug released on about Day 7 (FIGS. 4 and 6), after which time the correlation seems to become insignificant or lost.

Implants in accordance with the present invention which are structured to have roughness values (Ra, Rq) ranging from about 1 to about 10 provide an initial drug release ranging from between about 1% to about 5% at day one, or within one day of, implantation, and between about 5% to about 15% at day seven, or within seven days of implantation. Without intending to be bound by any particular theory of operation, it is believed that when roughness values (Ra, Rq) approach about 20, initial drug release ranging between about 10% and about 25% can be achieved.

In addition to an appropriate selection of porosity and/or roughness of the element, selection of an effective size and shape of elements 20 and 120 can be used to further control the rate of release, period of treatment and drug concentration in the eye.

Elements 20 and 120 in accordance with the present invention will have a controlled porosity and/or controlled roughness selected to enhance effectiveness of the system 10 and 110 with respect to the type of condition being treated, the amount of therapeutic agent necessary for treatment of the condition, the desired length of the treatment, and the mode of administering the treatment (e.g. whether implantation is accomplished by injection with a needle, surgical implantation, forceps, trocar, or the like).

For example, element 20 may comprise an extruded filament or rod having a size of between about 50 μm diameter and about 1 mm length, and about 500 μm diameter and about 6 mm length for administration or injection with a needle, and greater diameters/lengths for administration by surgical implantation. In one particular embodiment of the invention, implants are provided each having a diameter of about 450 μm and a length of about 6 mm.

The systems 10 and 110 of the present invention may be manufactured by any suitable technique that is capable of producing the element having a controlled porosity and/or controlled roughness as described elsewhere herein.

Porosity and/or roughness of the element 20 may be controlled by any suitable means. For example, porosity and/or roughness of a particular element can be selected and controlled by appropriate selection of extrusion parameters, for example, among other things, nozzle geometry, nozzle surface finish, extrusion temperature, extrusion rate or speed, for example, feed rate and screw speed, pressure, manner of cooling the extrudate, post-extrusion treatment, and the like. In addition, the composition of the precursor material for forming the elements of the invention will also affect porosity and roughness and thus can be selected to achieve a desired result.

In some situations, the system 10 of the invention comprises a plurality of such elements 20 having the same or different size and/or shape, each employing the same or different therapeutic agent, and the same or different release rates including burst effect release rates as controlled by varying porosities and/or surface roughness of the elements. For example, 2, 3, 4 or more elements in accordance with the present invention may be utilized. In this way, in a single administration a course of drug treatment may be achieved, where the pattern of release may be greatly varied. For example, a biphasic or triphasic release profile may be achieved with a single administration of a plurality of elements in accordance with the present invention.

Various techniques may be employed to produce the elements described and shown herein. Preferably the elements are produced by extrusion. However, other useful techniques include, but are not necessarily limited to, co-extrusion methods, injection molding, carver press methods, die cutting methods, heat compression, combinations thereof and the like. Techniques for producing the therapeutic component distributed within the matrix material include, but are not necessarily limited to, solvent-evaporation methods, phase separation methods, interfacial methods and the like.

The examples included herein are to illustrate certain aspects of the invention and are not to be considered to limit the scope of the invention.

### EXAMPLE I

Rates of release of the drug dexamethasone from implants that are substantially non-porous, and implants that have a controlled porosity in accordance with the present invention are measured and compared.

The first implants are made with dexamethasone and polylactic acid/polyglycolic acid copolymer. Dexamethasone powder and a powder of polylactic acid/polyglycolic acid (PLGA) copolymer having a relative average molecular weight of 15-20 kiloDaltons are mixed thoroughly at a ratio of about 50/50. The well mixed powder is filled into an extruder, heated for about 1 hour at about 95° C., and then extruded through a 20 gauge orifice.

Six implants are cut from the extrusion for study and drug release assessment. Scanning electron microscope images show that these first implants have little or no observable porosity.

The “infinite sink” method is used to measure the rate of drug release from the implants. Each individual first implant is placed in a glass vial filled with a receptor medium (9% NaCl in water). To allow for “infinite sink” conditions, the receptor medium volume is selected so that the concentration would not exceed 5% of saturation. Each of the glass vials is placed in a shaking water bath at about 37° C. Samples are taken for HPLC analysis from each vial at defined time points. Concentration values are used to calculate a cumulative release profile.

The release profile shows that the drug release is significantly slow with these first implants. Appreciable drug release does not begin until about the fourth week after initiation.

Second and third implants was manufactured using a twin screw extruder. Extrusion parameters are modified so as to produce six second implants having a porosity defined by relatively large, closely spaced pores disposed throughout the implants, and six third implants having a porosity defined by relatively small, spaced apart pores and micropores disposed throughout the implants. Scanning electron microscope images are used to confirm the nature of the porosity of the second and third implants.

The release rate of the drug from each second and third implant is determined using the same method as for the first (nonporous) implants.

It becomes apparent that with the inclusion of the large, closely spaced pores throughout the second implants, there is a pronounced increase in the rate of release of the drug. With the addition of small, spaced apart pores throughout the third implants, there is a marked increase in the rate of release of the drug relative to the first implants that are non-porous. In addition, there appears to be less of a delay in the initial release of the drug from the third implants relative to the first implants. In comparison to the second implants, the third implants show a more extended release rate.

This example illustrates that by controlling the porosity of the implant, the drug release rate of the implant can also be controlled.

The element may include a single therapeutic agent or a plurality of different therapeutic agents depending upon the nature of the condition or conditions of the eye being treated. The site of implantation of the element of the invention can vary depending upon the ocular condition being treated and the desired course of treatment.

For example, the present systems may be structured for treatment of an inflammation mediated condition, for example, uveitis. In this case, the therapeutic component may comprise an anti-inflammatory agent, for example, dexamethasone, and is preferably placed proximal to the uveal structures.

For example, the present systems may be structured for treatment of glaucoma. The element may be structured to provide sustained release of one or more neuroprotective agents that protect cells from excitotoxic damage. The element may be structured to be effective in delivering one or more beta-blockers, for example Timolol Maleate, to the eye on a substantially consistent basis. Other agents include N-methyl-D-aspartate (NMDA) antagonists, cytokines, and neurotrophic factors, preferably delivered intravitreally.

For example, the present systems may be structured for treatment of diabetic retinopathy. The therapeutic component may comprise one or more anti-angiogenic agents and/or one or more neurotropic agents, and may be structured to be implanted within the vitreous.

The present systems may be structured for treating age-related macular degeneration. For example, elements are provided for delivery of one or more neurotrophic factors intraocularly, preferably to the vitreous, and/or one or more anti-angiogenic factors intraocularly or periocularly, preferably periocularly, most preferably to the sub-Tenon's region.

The present invention also provides methods of treating an eye, wherein the methods generally comprises the step of placing the drug delivery systems described and shown elsewhere herein, into an eye, for example, using any suitable implantation method.

For example, the method may comprise implanting the elements 20, 120, at various sites in the eye. Suitable sites for implantation in the eye include the anterior chamber, posterior chamber, vitreous cavity, suprachoroidal space, subconjunctiva, episcleral, intracorneal, epicorneal and sclera. Suitable sites extrinsic to the vitreous comprise the suprachoroidal space, the pars plana and the like. The suprachoroid is a potential space lying between the inner scleral wall and the apposing choroid. Elements in accordance with the present invention that are introduced into the suprachoroid may deliver drugs to the choroid and to the anatomically apposed retina, depending upon the diffusion of the drug from the implant, the concentration of drug comprised in the implant and the like.

The elements may be introduced over or into an avascular region. The avascular region may be naturally occurring, such as the pars plana, or a region made to be avascular by surgical methods. Surgically-induced avascular regions may be produced in an eye by methods known in the art such as laser ablation, photocoagulation, cryotherapy, heat coagulation, cauterization and the like. It may be particularly desirable to produce such an avascular region over or near the desired site of treatment, particularly where the desired site of treatment is distant from the pars plana or placement of the element at the pars plana is not possible. Introduction of the over an avascular region will allow for diffusion of the drug from the element and into the inner eye and avoids diffusion of the drug into the bloodstream.

This may be more clearly understood with reference to FIG. 8, which depicts a cross-sectional view of a human eye 300 in order to illustrate the various sites that may be suitable for implantation of the elements in accordance with the present invention.

The eye 300 comprises a lens 316 and encompasses the vitreous chamber 318. Adjacent to the vitreous chamber is the optic part of the retina 322. Implantation may be into the vitreous 318, intraretinal 322 or subretinal 324. The retina 322 is surrounded by the choroid 326. Implantation may be intrachoroidal or suprachoroidal 328. Between the optic part of the retina and the lens, adjacent to the vitreous, is the pars plana 330. Surrounding the choroid 326 is the sclera 332. Implantation may be intrascleral 332 or episcleral 334. The external surface of the eye is the cornea 342. Implantation may be epicorneal 342 or intra-corneal 344. The internal surface of the eye is the conjunctiva 346. Behind the cornea is the anterior chamber 348, behind which is the lens 316. The posterior chamber 352 surrounds the lens, as shown in the figure. Opposite from the external surface is the optic nerves, and the arteries and vein of the retina. Implants into the meningeal spaces 358, the optic nerve 360 and the intraoptic nerve 361 allows for drug delivery into the central nervous system, and provide a mechanism whereby the blood-brain barrier may be crossed.

Other sites of implantation include the delivery of anti-tumor drugs to neoplastic lesions, e.g. tumor, or lesion area, e.g. surrounding tissues, or in those situations where the tumor mass has been removed, tissue adjacent to the previously removed tumor and/or into the cavity remaining after removal of the tumor. The implants may be administered in a variety of ways, including surgical means, injection, trocar, etc.

Among the diseases/conditions which can be treated or addressed in accordance with the present invention include, without limitation, the following:

MACULOPATHIES/RETINAL DEGENERATION: Non-Exudative Age Related Macular Degeneration (ARMD), Exudative Age Related Macular Degeneration (ARMD), Choroidal Neovascularization, Diabetic Retinopathy, Acute Macular Neuroretinopathy, Central Serous Chorioretinopathy, Cystoid Macular Edema, Diabetic Macular Edema.

UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, Vogt-Koyanagi-Harada Syndrome.

VASCULAR DISEASES/EXUDATIVE DISEASES: Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angitis, Sickle Cell Retinopathy and other Hemoglobinopathies, Angioid Streaks, Familial Exudative Vitreoretinopathy, Eales Disease.

TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy.

PROLIFERATIVE DISORDERS: Proliferative Vitreal Retinopathy and Epiretinal Membranes, Proliferative Diabetic Retinopathy.

INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis.

GENETIC DISORDERS: Retinitis Pigmentosa, Systemic Disorders with Accosiated Retinal Dystrophies, Congenital Stationary Night Blindness, Cone Dystrophies, Stargardt's Disease and Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum.

RETINAL TEARS/HOLES: Retinal Detachment, Macular Hole, Giant Retinal Tear.

TUMORS: Retinal Disease Associated with Tumors, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

MISCELLANEOUS: Punctate Inner Choroidopathy, Acute Posterior Multifocal Placoid Pigment Epitheliopathy, Myopic Retinal Degeneration, Acute Retinal Pigment Epithelitis and the like.

While this invention has been described with respect to various specific examples and embodiments, it is to be understood that the invention is not limited thereto and that it can be variously practiced within the scope of the following claims.

1. A drug delivery system for controlled drug release into an eye comprising:
an element sized and adapted for placement into an eye, said element including a therapeutic component and a matrix component, the therapeutic component being located in combination with the matrix component, the element having at least one of a controlled porosity and a controlled roughness effective in controlling a release rate of the therapeutic component from the element into an eye in which the element is placed.

2. The system of claim 1 wherein the at least one of a controlled porosity and a controlled roughness is effective in controlling the release rate of the therapeutic component from the element into an eye in which the element is placed for a period of time of less than about 14 days after placement in the eye.

3. The system of claim 1 wherein the at least one of a controlled porosity and a controlled roughness is effective in controlling the release rate of the therapeutic component from the element into an eye in which the element is placed for a period of time of less than about 10 days after placement in the eye.

4. The system of claim 1 wherein the at least one of a controlled porosity and a controlled roughness is effective in controlling the release rate of the therapeutic component from the element into an eye in which the element is placed for a period of time of less than about 7 days after placement in the eye.

5. The system of claim 1 wherein the therapeutic component is distributed substantially uniformly throughout the matrix component.

6. The system of claim 1 wherein the element has a controlled porosity and an increase in porosity of the element is effective in increasing the release rate of the therapeutic component from the element into an eye in which the element is placed.

7. The system of claim 1 wherein the element has a controlled roughness and an increase in roughness of the element is effective in increasing the release rate of the therapeutic component from the element into an eye in which the element is placed.

8. The system of claim 1 wherein the at least one of the controlled porosity and the controlled roughness is effective in releasing between about 1% to about 25% of the therapeutic component from the element within about one day of the element being placed in an eye.

9. The system of claim 1 wherein the at least one of the controlled porosity and the controlled roughness is effective in releasing between about 5% to about 20% of the therapeutic component from the element within about one day of the element being placed in an eye.

10. The system of claim 1 wherein the at least one of the controlled porosity and the controlled roughness is effective in releasing between about 10% to about 15% of the therapeutic component from the element within about one day of the element being placed in an eye.

11. The system of claim 1 wherein the at least one of the controlled porosity and the controlled roughness is effective in releasing between about 1 % to about 25% of the therapeutic component from the element within about 7 days of the element being placed in an eye.

12. The system of claim 1 wherein the at least one of the controlled porosity and the controlled roughness is effective in releasing between about 5% to about 20% of the therapeutic component from the element within about 7 days of the element being placed in an eye.

13. The system of claim 1 wherein the at least one of the controlled porosity and the controlled roughness is effective in releasing between about 10% to about 15% of the therapeutic component from the element within about 7 days of the element being placed in an eye.

14. The system of claim 1 wherein at least a portion of the element is biodegradable.

15. The system of claim 1 wherein the matrix component includes a substantially biodegradable material.

16. The system of claim 1 wherein the therapeutic component is selected from the group consisting of cortisone, dexamethasone, fluocinolone, hydrocortisone, methylprednisolone, prednisolone, prednisone, and triamcinolone, and their derivatives.

17. The system of claim 1 wherein the matrix component comprises a polymeric material.

18. The system of claim 1 wherein the therapeutic component is selected from the group consisting of corticosteroids and mixtures thereof.

19. The system of claim 1 wherein the therapeutic component is dexamethasone.

20. The system of claim 1 wherein the matrix component includes a polymeric material including a polymer selected from the group consisting of poly-lactic acid, poly glycolic acid, copolymers of lactic acid and glycolic acid and mixtures thereof.

21. The system of claim 1 wherein the material component includes a polymeric material selected from the group consisting of copolymers of lactic acid and glycolic acid, and mixtures thereof.

22. A method of treating an eye comprising placing the drug delivery system of claim 1 into an eye.

23. The system of claim 1 wherein the matrix component has a controlled roughness and an increase in roughness of the element is effective in increasing the release rate of the therapeutic component from the element into an eye in which the element is placed.

24. A method of making a drug delivery system for modified drug delivery into an eye comprising:
forming an element sized and adapted for placement into an eye, said element including a therapeutic component and a matrix component, the therapeutic component being located in the matrix component, wherein the forming step is conducted at conditions effective in controlling at least one of a porosity of the matrix component and a roughness of the matrix component, in order to provide a controlled release rate of the therapeutic component from the element into an eye in which the element is placed.

25. The method of claim 24 wherein the therapeutic component is distributed in the matrix component.

26. The system of claim 24 wherein the at least one of the controlled porosity and the controlled roughness is effective in releasing between about 1 % to about 25% of the therapeutic component from the element within about one day of the element being placed in an eye.

27. The system of claim 24 wherein the at least one of the controlled porosity and the controlled roughness is effective in releasing between about 5% to about 20% of the therapeutic component from the element within about one day of the element being placed in an eye.

28. The system of claim 24 wherein the at least one of the controlled porosity and the controlled roughness is effective in releasing between about 1 % to about 25% of the therapeutic component from the element within about 7 days of the element being placed in an eye.

29. The system of claim 24 wherein the at least one of the controlled porosity and the controlled roughness is effective in releasing between about 5% to about 20% of the therapeutic component from the element within about 7 days of the element being placed in an eye.

30. The method of claim 24 wherein the matrix component includes a polymeric material.

31. The method of claim 24 wherein the therapeutic component is selected from the group consisting of cortisone, dexamethasone, fluocinolone, hydrocortisone, methylprednisolone, prednisolone, prednisone, and triamcinolone, and their derivatives.

32. The method of claim 24 wherein the therapeutic component is selected from the group consisting of corticosteroids and mixtures thereof.

33. The method of claim 24 wherein the therapeutic component is dexamethasone.

34. The method of claim 24 wherein the matrix component includes a polymeric material selected from the group consisting of copolymers of lactic acid and glycolic acid, and mixtures thereof.

35. The method of claim 24 wherein said forming step includes extruding a combination of the matrix component and the therapeutic component.
